(19) United States
(12) Patent Application Publication
(10) Pub. No.: US 2007/0129439 A1
(43) Pub. Date: Jun. 7, 2007

(54) COMPOSITIONS CONTAINING SUBSTITUTED QUINOLINES AND SUBSTITUTED DIPHENYL SULFONES AND METHODS OF USE

(75) Inventors: Dana J. Giulian, Houston, TX (US); David P. Jacobus, Princeton, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Baylor College of Medicine

(21) Appl. No.: 11/671,372

(22) Filed: Feb. 5, 2007

Related U.S. Application Data
(63) Continuation of application No. 10/495,553, filed on Jan. 18, 2005.
(60) Provisional application No. 60/443,219, filed on Jan. 27, 2003.

(51) Int. Cl. A61K 31/195; A61K 31/192; A61K 31/135

(52) U.S. Cl. 514/562; 514/646; 514/568

(57) ABSTRACT

Combination therapies of substituted quinolines and substituted diphenyl sulfones are disclosed. More specifically, compositions containing substituted quinolines and substituted diphenyl sulfones are disclosed. In addition, methods of using the compositions in the treatment of neurodegenerative disorders, including, inter alia, Alzheimer's dementia, HIV-1 associated dementia, and Creutzfeld-Jakob disease are also disclosed.

### CROSS REFERENCE TO RELATED APPLICATIONS

This application is a continuation of application Ser. No. 10/495,553, filed Jan. 18, 2005, which is a National Phase Application of PCT/US04/02147, filed Jan. 27, 2004, which claims the benefit of application Ser. No. 60/443,219, filed Jan. 27, 2003, the disclosures of which are incorporated by reference in their entireties.

### REFERENCE TO GOVERNMENT GRANTS

Portions of the disclosure herein may have been supported in part by grants from the National Institutes of Health, Grant No. NS25637, AG12548, and NS34000. The United States Government may have certain rights in this application.

### FIELD OF THE INVENTION

The present invention generally relates to combination therapies of substituted quinolines and substituted diphenyl sulfones. More specifically, the present invention relates to compositions containing substituted quinolines and substituted diphenyl sulfones and methods of using the compositions in the treatment of neurodegenerative disorders, including, inter alia, Alzheimer's dementia, HIV-1 associated dementia, or Creutzfeld-Jakob disease.

### BACKGROUND OF THE INVENTION

Alzheimer's disease is a slowly progressive, neurodegenerative disorder that involves abnormalities in β-amyloid (Aβ) metabolism, the formation of β-amyloid plaques, chronic neuroinflammation, and loss of synapses and neurons in the neocortex and hippocampus. It is generally believed that the loss of synapses and neurons account for the severe defects in cognition associated with Alzheimer's disease.

Neuroinflammation and neuron loss is believed to be involved in several neurodegenerative disorders, including Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome, and the like.

Although much effort has been expended to develop therapeutics for Alzheimer's disease and other neurodegenerative diseases associated with neuroinflammation and neuron loss, little progress has been made in identifying agents that provide neuroprotection.

Therefore, it would be desirable to identify neuroprotective agents useful for the prevention and treatment of neurodegenerative disorders, especially those associated with neuroinflammation, neuron loss, and cognitive loss. The compositions and methods of the present invention are directed toward these, as well as other, important ends.

### SUMMARY OF THE INVENTION

Accordingly, the present invention is directed, in part, to compositions and methods useful for the prevention and treatment of neurodegenerative disorders, especially those associated with neuroinflammation, neuron loss, and cognitive loss.

In one aspect, the invention is directed to pharmaceutical compositions, comprising:
at least one substituted quinoline or pharmaceutically-acceptable salt or enantiomer or prodrug thereof; and
at least one compound of formula I: 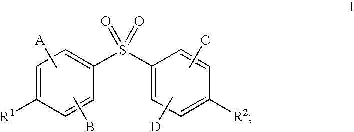
or pharmaceutically-acceptable salt or prodrug thereof;
wherein:
A, B, C, and D are, independently, H, lower alkyl, cyano, OR⁵, —C(═O)OR⁵, SR⁶, halo, SO₂R⁶, NR⁶R⁷, —SO₂NR⁶R⁷;
R¹ and R² are, independently, NH₂, NHC(═O)R³, or —N═NR⁴;
R³ is H, lower alkyl, -alkyl-OR⁵, -alkyl-C(═O)OR⁵, or -alkyl-C(═O)NHR⁵;
R⁴ is 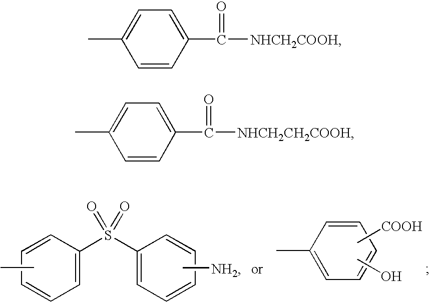
R⁵ is hydrogen, lower alkyl, substituted lower alkyl, lower alkenyl, substituted lower alkenyl, lower alkynyl, substituted lower alkynyl, aryl, haloaryl, substituted aryl, acyl, or heterocyclyl;
R6 is independently hydrogen, alkyl, substituted lower alkyl, lower alkenyl, substituted lower alkenyl, lower alkynyl, substituted lower alkynyl, alkyl substituted aryl, or acyl; and
R⁷ is independently hydrogen, alkyl, substituted lower alkyl, lower alkenyl, lower alkynyl, substituted lower alkenyl, substituted lower alkynyl, alkyl substituted aryl, acyl, —SO₂R⁵, or SO₂NR⁵R⁶.

In another aspect, the invention is directed to method of treating a disorder associated with neuron loss, comprising the step of:
administering to a patient in need thereof an effective amount of the composition, comprising:
at least one substituted quinoline or pharmaceutically-acceptable salt or enantiomer or prodrug thereof; and
at least one compound of formula I or pharmaceutically-acceptable salt or prodrug thereof, as defined above.
   The disorders associated with neuron loss include, inter alia, Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome.

In yet another aspect, the invention is directed to methods of treating a neurodegenerative disorder, comprising the step of:
administering to a patient in need thereof an effective amount of the composition, comprising:
at least one substituted quinoline or pharmaceutically-acceptable salt or enantiomer or prodrug thereof; and
at least one compound of formula I or pharmaceutically-acceptable salt or prodrug thereof, as defined above.
   The neurodegenerative disorders include, inter alia, Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome.

In further aspect, the invention is directed to methods of treating a patient at risk of cognitive loss, comprising the step of:
administering to said patient an effective amount of the composition, comprising:
at least one substituted quinoline or pharmaceutically-acceptable salt or enantiomer or prodrug thereof; and
at least one compound of formula I or pharmaceutically-acceptable salt or prodrug thereof, as defined above.
   Such patients may be afflicted with mild cognitive impairment, mild cognitive motor dysfunction, HIV-associated dementia, neuro-AIDS, prion disease, acute stroke or acute trauma.

### BRIEF DESCRIPTION OF THE DRAWINGS

FIG. 1 is a plot of % neuron loss for several test agents in a rat model with Alzheimer's disease (AD).

FIG. 2 is a plot of % neuron loss as a function of drug concentration for several test agents.

FIG. 3 is an isobologram of hydroxychloroquine and dapsone.

FIG. 4 shows improvement in Alzheimer cognition by cumulative T scores for cognitive test battery (six month clinical trial).

FIG. 5 shows improvement in HIV cognition with combined drug product of the invention (six week clinical trial).

### DETAILED DESCRIPTION OF THE INVENTION

The present invention is generally directed to combinations therapies for the prevention and/or treatment of neurodegenerative disorders, especially those associated with neuroinflammation and neuron loss. In one aspect, the invention is directed to compositions comprising substituted quinolines and substituted diphenyl sulfones. In another aspect, the invention is directed to the use of such compositions in the prevention and/or treatment of neurodegenerative disorders, including, inter alia, Alzheimer's dementia, HIV-1 associated dementia, and Creutzfeld-Jakob disease.

As employed above and throughout the disclosure, the following terms, unless otherwise indicated, shall be understood to have the following meanings.

As used herein and in the appended claims, the singular forms “a,” “an,” and “the” include the plural reference unless the context clearly indicates otherwise.

As used herein, “halo” refers to —F, —Cl, or —Br.

As used herein, “alkyl” refers to a saturated straight, branched, cyclic, or multicyclic hydrocarbon having from 1 to about 20 carbon atoms (and all combinations and subcombinations of ranges and specific numbers of carbon atoms therein). The term “lower alkyl” herein refers to those alkyl groups having from about 1 to about 10 carbon atoms, these being preferred. Alkyl groups include, but are not limited to, methyl, ethyl, n-propyl, isopropyl, cyclopropyl, n-butyl, isobutyl, t-butyl, cyclobutyl, n-pentyl, cyclopentyl, isopentyl, neopentyl, n-hexyl, isohexyl, cyclohexyl, cycloheptyl, cyclooctyl, decahydronaphthalenyl, adamantyl, 3-methylpentyl, 2,2-dimethylbutyl, and 2,3-dimethylbutyl. Alkyl groups can be substituted or unsubstituted.

As used herein, “haloalkyl” means an alkyl group substituted with one or more halo groups selected from —F or —Cl.

As used herein, “alkoxy” means an alkyl-O— moiety, wherein “alkyl” as defined above.

As used herein, “haloalkoxy” means an alkoxy group substituted with one or more halo groups selected from —F and —Cl.

As used herein, “alkenyl” refers to an alkyl group having one or more double bonds. The term “lower alkenyl” herein refers to those alkenyl groups having from about 2 to about 10 carbon atoms.

As used herein, “alkynyl” refers to an alkyl group having one or more triple bonds. The term “lower alkynyl” herein refers to those alkynyl groups having from about 2 to about 10 carbon atoms.

As used herein, “aryl” refers to a mono-, di-, tri-, or other multicyclic aromatic ring system having from about 5 to about 30 carbon atoms (and all combinations and subcombinations of ranges and specific numbers of carbon atoms therein), with from about 6 to about 14 carbons being preferred. Non-limiting examples include phenyl, naphthyl, anthracenyl, and phenanthrenyl. Aryl groups can be substituted or unsubstituted.

As used herein, “haloaryl” refers to means an aryl group substituted with one or more halo groups selected from —F, —Cl, and —Br.

As used herein, “aralkyl” or “arylalkyl” refers to aryl-substituted alkyl radicals having from about 6 to about 50 carbon atoms (and all combinations and subcombinations of ranges and specific numbers of carbon atoms therein), with from about 6 to about 20 carbon atoms being preferred. Non-limiting examples include, for example, benzyl, phenylethyl, 3-phenylprop-1-yl, tetrahydronaphthalenyl, 3-phenylprop-2-yl, and 4-naphthylhex-1-yl. Aralkyl groups can be substituted or unsubstituted. Substitution may occur on the aryl ring carbons or alkyl carbons of the aralkyl.

As used herein, “heteroaryl” refers to a mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrrolyl, furyl, pyridyl, 1,2,4-thiadiazolyl, pyrimidinyl, isothiazolyl, thiazolyl, triazolyl, imidazolyl, tetrazolyl, pyrazinyl, quinolyl, isoquinolyl, thiophenyl, benzothienyl, isobenzofuryl, pyrazolyl, indolyl, purinyl, carbazolyl, benzimidazolyl, oxazolyl, and isoxazolyl. Heteroaryl groups can be substituted or unsubstituted.

As used herein, “acyl” refers to an alkyl-C(═O)— or an aryl-C(═O)— group.

Typically, substituted chemical moieties include one or more substituents that replace hydrogen. Exemplary substituents include, for example, halo (e.g., —F, —Cl, —Br), (provided that when halo is —Br, the —Br is attached to an Sp² carbon such as on a carbon of an alkenyl or a ring carbon of aryl or heteroaryl group), alkoxy, haloalkoxy, —OCF₃, alkylthio, monohaloalkylthio, polyhaloalkylthio, —SCF₃, alkyl, —CF₃, haloalkyl, lower alkyl, spiroalkyl, alkenyl, alkynyl, aralkyl, aryl, heteroaryl, heterocyclyl, hydroxyl (—OH), nitro (—NO₂), cyano (—CN), sulfonyl (—SO₂R⁴), sulfamoyl (—SO₂NR⁵R⁶), —SR⁴, amino (—NH₂, NHR⁵, NHR⁶, N(R⁵R⁶) and the like.

As used herein, “side effect” refers to a consequence other than the one(s) for which an agent or measure is used, as the adverse effects produced by a drug, especially on a tissue or organ system other then the one sought to be benefited by its administration. In the case, for example, of agents for the prevention or treatment of neuron loss or neurological disorders, the term “side effect” may preferably refer to such conditions as, for example, erythocytic and gastrointestinal effects.

As used herein, “effective amount” refers to an amount of a compound as described herein that may be effective to inhibit, or treat the symptoms of particular disease, disorder, or side effect, or to prevent, inhibit, or diminish the onset of the symptoms of particular disease, disorder, or side effect. Such diseases, disorders, and side effects include, but are not limited to, Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome.

As used herein, “treating” refers to the preventative, curative, and palliative treatment of a condition, and includes, in particular, not only the prevention and/or treatment of a condition per se, but also the prevention of the progression of a condition, such as, for example, the progression of Alzheimer's dementia.

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

As used herein, “prodrug” is intended to include any covalently bonded carriers that release the active parent drug or whose form is converted, for example, as according to formula I, formula II, or formula III or other formulas or compounds employed in the methods of the present invention such as dapsone, in vivo when such prodrug is administered to a patient. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs.

“Patient” refers to an animal, including a mammal, preferably a human.

Accordingly, the present invention is directed, in part, to pharmaceutical compositions, comprising:
at least one substituted quinoline or pharmaceutically-acceptable salt or enantiomer or prodrug thereof; and
at least one compound of formula I: 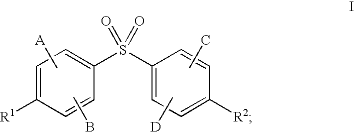
or pharmaceutically-acceptable salt or prodrug thereof;
wherein:
A, B, C, and D are, independently, H, lower alkyl, cyano, OR⁵, —C(═O)OR⁵, SR⁶, halo, SO₂R⁶, NR⁶R⁷, —SO₂NR⁶R⁷;
R¹ and R² are, independently, NH₂, NHC(═O)R³, or —N═NR⁴;
R³ is H, lower alkyl, -alkyl-OR⁵, -alkyl-C(═O)OR⁵, or -alkyl-C(═O)NHR⁵;
R⁴ is 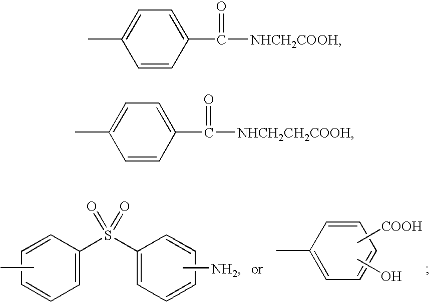
R⁵ is hydrogen, lower alkyl, substituted lower alkyl, lower alkenyl, substituted lower alkenyl, lower alkynyl, substituted lower alkynyl, aryl, haloaryl, substituted aryl, acyl, or heterocyclyl;
R⁶ is independently hydrogen, alkyl, substituted lower alkyl, lower alkenyl, substituted lower alkenyl, lower alkynyl, substituted lower alkynyl, alkyl substituted aryl, or acyl; and
R⁷ is independently hydrogen, alkyl, substituted lower alkyl, lower alkenyl, lower alkynyl, substituted lower alkenyl, substituted lower alkynyl, alkyl substituted aryl, acyl, —SO₂R⁵, or SO₂NR⁵R⁶.

Hydroxychloroquine, a substituted quinoline, and dapsone, a substituted diphenyl sulfones have been used individually at high levels to prevent and/or treat neurological disorders, including Alzheimer's dementia. However, it has been unexpectedly discovered that, when combined, a substituted quinoline and a substituted diphenyl work synergistically to prevent and treat loss of neurons and hence are useful in the prevention and treatment of neurological disorders, including, inter alia, Alzheimer's dementia, HIV-1 associated dementia, and Creutzfeld-Jakob disease, at lower levels.

The substituted quinoline and the substituted diphenyl sulfones of the present invention may be prepared in a number of ways well known to those skilled in the art. The compounds can be synthesized, for example, by the methods described in the references listed below or variations thereon as appreciated by the skilled artisan. All processes disclosed in association with the present invention are contemplated to be practiced on any scale, including milligram, gram, multigram, kilogram, multikilogram or commercial industrial scale.

The substituted quinolines useful in the invention may be prepared by synthetic techniques that are well known in the art. See, for example, Surrey, Hammer, J. Am. Chem. Soc., 72, 1814, (1950) and U.S. Pat. No. 2,546,658, the disclosures of which are herein incorporated by reference. Most of the substituted quinolines are commercially-available.

The substituted diphenyl sulfone compounds of formula I useful in the invention may be prepared by synthetic techniques that are well known in the art. See, for example, U.S. Pat. No. 3,689,671; U.S. Pat. No. 3,702,362; U.S. Pat. No. 3,715,375; U.S. Pat. No. 3,775,403; U.S. Pat. No. 3,775,444; U.S. Pat. 3,786,050, and U.S. Pat. No. 4,338,334; H. Heyman and L. F. Fieser, Journal of the American Chemical Society, 87, 1979, (1945), the disclosures of which are herein incorporated by reference. Most of the substituted diphenyl sulfones are commercially-available. For example, dapsone (4,4′-diaminodiphenylsulfone) is available from Jacobus Pharmaceuticals Company, Inc.

In certain preferred embodiments, suitable substituted quinolines include 4-aminoquinoline, 8-aminoquinoline, and hydroxymethylquinoline, or pharmaceutically acceptable salt or enantiomer or prodrug thereof. More preferred substituted quinolines include hydroxychloroquine, chloroquine, amodiaquine, amopyroquine, cycloquine, oxychloroquine, sontoquine, amodiaquine, primaquine, mefloquine, quinacrine, quinine, thalidomide, sulfasalazine, and sulfapyridine, or pharmaceutically acceptable salt or enantiomer or prodrug thereof. Even more preferred substituted quinolines include hydroxychloroquine or chloroquine, or pharmaceutically acceptable salt or enantiomer or prodrug thereof. A particularly preferred substituted quinoline is hydroxychloroquine or pharmaceutically acceptable salt or enantiomer or prodrug thereof.

In certain preferred embodiments, the pharmaceutical composition comprises the compound of formula I,
wherein A, B, C, and D are each H.

In certain preferred embodiments, the pharmaceutical composition comprises the compound of formula I,
wherein R¹ and R² are, independently, NH₂ or NHC(═O)R³, preferably where R³ is methyl.
   In even more preferred embodiments, R¹ and R2 are each NH₂.

In certain preferred embodiments, R⁴ is

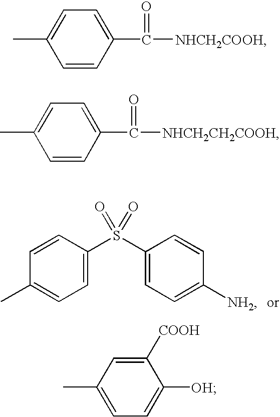

In certain preferred embodiments, the pharmaceutical composition comprises a compound of formula II: 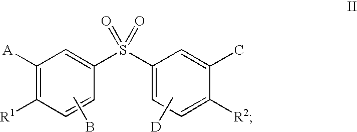
or pharmaceutically-acceptable salt thereof.

In certain preferred embodiments, the pharmaceutical composition comprises a compound of formula III; 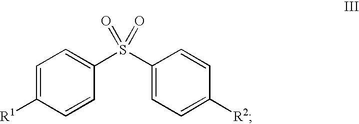
or pharmaceutically-acceptable salt thereof.

In certain preferred embodiments, the pharmaceutical composition comprises a compound of the formula: 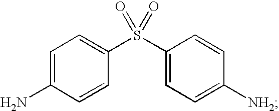
or pharmaceutically-acceptable salt thereof.
   This compound is also known as dapsone.

In certain preferred embodiments, the pharmaceutical composition comprises a compound of one of the following formulae, each of which is known prodrug of dapsone, described above: 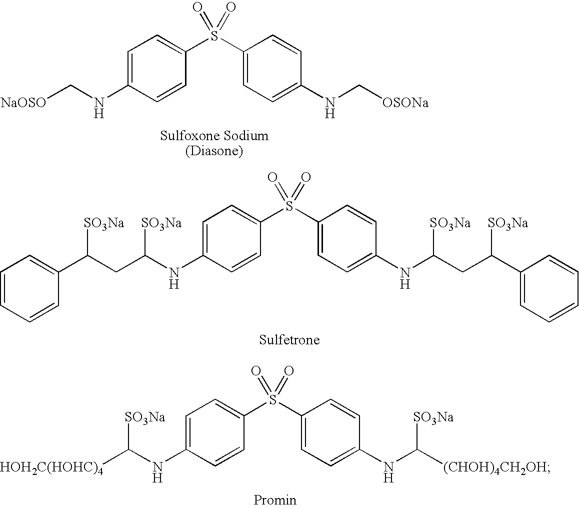
or pharmaceutical salt thereof.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form or to any specific optical or geometric isomer.

When any variable occurs more than one time in any constituent or in any formula, its definition in each occurrence is independent of its definition at every other occurrence. Combinations of substituents and/or variables are permissible only if such combinations result in stable compounds. It is further understood that, while certain substituents are minimally required, the moiety may be further substituted with the same substituent(s), another substituent(s) from the group of required substituents, or other substituent(s) not from the group of required substituents.

All forms of the compounds useful in the pharmaceutical compositions of the invention, including free acid, free base, and zwitterions, isomorphic crystalline forms, all chiral and racemic forms, hydrates, solvates, and acid salt hydrates, are contemplated to be within the scope of the present invention.

Compounds of the pharmaceutical compositions of the invention may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

The compounds of the present invention may be made in the form of the monohydrohalic acid addition salts and/or the solvated compound, for example the hydrochloride hydrate or the hydrobromide. Other salts may be made however by simple reaction of a base with acid and may be desirable in order to modify the properties of the product, such as its toxicity, taste, physical form, or rate of release into the body. For example, the compounds may be made in the form of the sulfate, bisulfate, phosphate, nitrate, acetate, maleate, phthalate, succinate, phosphate, nitrobenzoate, stearate, mandelate, N-acetyl-glycinate, pamoate, sulfonate, di-sulfonate, cyclohexyl sulfamate, citrate, tartrate, propionate, glycolate, lactate, malate, ascorbate, hydroxymaleate, phenylacetate, glutamate, benzoate, salicylate, sulfanilate, 2-acetoxybenzoate, fumarate, toluenesulfonate, methanesulfonate, ethane disulfonate, isethionate, mesylate or gluconate, and the like.

The compounds (either the substituted quinolines or the substituted diphenyl sulfones) of the pharmaceutical compositions of the invention may exist in prodrug form. Prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

In some embodiments, the pharmaceutical compositions of the invention further comprise a pharmaceutically acceptable carrier or diluent. Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers and/or diluents are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Oral formulations are preferred. Formulations for oral or injected use are based on sufficient solubility as to allow the therapeutic agent to enter solution in the stomach or in an injectable medium. Suitable drug formulations include, but are not limited to, tablets, pills, capsules, sachets, granules, powders, chewing gums, suspensions, emulsions, suppositories, and solutions. Particularly preferred for oral use are tablets and capsules of all varieties and microbe-free solutions for injection or infusion. Where appropriate and necessary the formulations may include diluents, binding agents, dispersing agents, surface-active agents, lubricating agents, coating materials, flavoring agents, coloring agents, controlled release formulations, sweeteners or any other pharmaceutically acceptable additives, for example, gelatin, sodium starch glycolate, lactose, starch, talc, magnesium stearate, microcrystalline cellulose, Povidone, hydrogenated or unsaturated oils, polyglycols, syrups or other aqueous solutions. Where the formulations are tablets or capsules and the like the formulations may be presented as premeasured unit doses or in multidose containers from which the appropriate unit dose may be withdrawn.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

The injectable form may be an aqueous or nonaqueous solution, suspension or emulsion in a pharmaceutically acceptable liquid, e.g. sterile pyrogen-free water or parenterally acceptable oils or mixture of liquids which may contain bacteriostatic agents, antioxidants or other preservatives and stabilizers, buffers (preferably but not limited to a physiological pH range of 6.5-7.7, solutes to render the solution isotonic with the blood, thickening agents, suspending agents or other pharmaceutically acceptable additives. Such forms will be presented in unit dose form such as ampules or disposable injection devices or in multi-dose forms such as a bottle from which the appropriate dose may be withdrawn, or as a solid form or concentrate that can be used to quickly prepare an injectable formulation. All formulations for injection are preferable as sterile and pyrogen free. Suppositories containing the compound will also contain suitable carriers, e.g. cocoa butter, polyglycols or other state-of-the-art carriers.

Preferably the pharmaceutical composition is in single unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

When the combination products are not formulated together in a single dosage form, the substituted quinoline and the substituted diphenyl sulfone may be administered at the same time or simultaneously (that is, together), or in any order. When not administered at the same time or simultaneously, that is, when administered sequentially, preferably the administration of a substituted quinoline and the substituted diphenyl sulfone occurs less than about one hour apart, more preferably less than about 30 minutes apart, even more preferably less than about 15 minutes apart, and still more preferably less than about 5 minutes apart.

In addition to standard pharmaceutical additives there may be included within formulations of the compound other therapeutic agents.

The dosage of the composition of the present invention that will be most suitable for prophylaxis or treatment will vary with the form of administration, the particular compounds chosen and the physiological characteristics of the particular patient under treatment. Generally, small dosages may be used initially and, if necessary, increased by small increments until the desired effect under the circumstances is reached. Generally speaking, oral administration may require higher dosages.

Preferably, administration of the combination products of the invention is oral, although other routes of administration, as described above, are contemplated to be within the scope of the present invention. Although it is preferable that the substituted quinoline and the substituted diphenyl sulfone are all administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Although the proper dosage of the pharmaceutical composition of this invention will be readily ascertainable by one skilled in the art, once armed with the present disclosure, by way of general guidance, typically a daily dosage may range from about 0.01 to about 100 milligrams of the substituted quinoline (and all combinations and subcombinations of ranges therein) and about 0.001 to about 100 milligrams of the substituted diphenyl sulfone (and all combinations and subcombinations of ranges therein) per kilogram of patient body weight. Preferably, the a daily dosage may be about 0.1 to about 10 milligrams of the substituted quinoline and about 0.1 to about 10 milligrams of the substituted diphenyl sulfone per kilogram of patient body weight. Even more preferably, the daily dosage may be about 1.0 to about 10.0 milligrams of the substituted quinoline and about 1 to about 4.0 milligrams of the substituted diphenyl sulfone per kilogram of patient body weight. With regard to a typical dosage form of this type of combination product, such as a tablet, the substituted quinoline generally may be present in an amount of about 100 to about 300 milligrams and the substituted diphenyl sulfone in an amount of about 25 to about 100 milligrams. The preferred dosage form of the combination product contains, preferably in tablet form, 50 mg of dapsone and 200 mg of hydroxychloroquine, wherein the combination product is administered to the patient two times a day (bid). Alternatively, the combination product may be administered once a day (os) or three times a day (tid).

Particularly when provided as a single dosage form, the potential exists for a chemical interaction between the combined active ingredients, i.e., the substituted quinoline and the substituted diphenyl sulfone. For this reason, the preferred dosage forms of the combination products of this invention are formulated such that although the active ingredients are combined in a single dosage form, the physical contact between the active ingredients is minimized (that is, reduced).

In order to minimize contact, one embodiment of this invention where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material that effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-pareils, which are then enteric coated. These enteric-coated microtablets, particles, granules or non-pareils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

In certain embodiments, the invention is directed to methods of treating a disorder associated with neuron loss, comprising the step of:
administering to a patient in need thereof an effective amount of the composition, comprising:
at least one substituted quinoline or pharmaceutically-acceptable salt or enantiomer or prodrug thereof; and at least one compound of formula I or pharmaceutically-acceptable salt or prodrug thereof, as defined above.
   The disorders associated with neuron loss include, inter alia, Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome. The method of the invention is particularly useful for the prevention and treatment of neuron loss associated with Alzheimer's dementia, HIV-1 associated dementia, and Creutzfeld-Jakob disease. In certain preferred embodiments, the composition is administered during the early progression of the neurodegenerative disorder.

In certain other embodiments, the invention is directed to methods of treating a neurodegenerative disorder, comprising the step of:
administering to a patient in need thereof an effective amount of the composition, comprising:
at least one substituted quinoline or pharmaceutically-acceptable salt or enantiomer or prodrug thereof; and
at least one compound of formula I or pharmaceutically-acceptable salt or prodrug thereof, as defined above.
   The disorders associated with neuron loss include, inter alia, Alzheimer's dementia, HIV-1 associated dementia, spongiform encephalopathy, Creutzfeld-Jakob disease, stroke, trauma, multiple sclerosis, Parkinson's disease, HIV infection of the central nervous system, hereditary hemorrhage with amyloidosis-Dutch type, cerebral amyloid angiopathy, or Down's syndrome. The method of the invention is particularly useful for the prevention and treatment of Alzheimer's dementia, HIV-1 associated dementia, and Creutzfeld-Jakob disease. In certain preferred embodiments, the composition is administered during the early progression of said neurodegenerative disorder.

In further aspect, the invention is directed to methods of treating a patient at risk of cognitive loss, comprising the step of:
administering to said patient an effective amount of the composition, comprising:
at least one substituted quinoline or pharmaceutically-acceptable salt or enantiomer or prodrug thereof; and
at least one compound of formula I or pharmaceutically-acceptable salt or prodrug thereof, as defined above.
   Such patients may be afflicted with mild cognitive impairment, mild cognitive motor dysfunction, HIV-associated dementia, neuro-AIDS, prion disease, acute stroke or acute trauma. In certain preferred embodiments, the composition is administered during the early progression of said cognitive loss.

The present invention is further defined in the following Examples, in which all parts and percentages are by weight, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

The compositions of the invention and comparative compounds and compositions were evaluated in accordance with the test methods described in U.S. Pat. No. 6,043,283; U.S. Pat. No. 6,071,493; U.S. Pat. No. 6,451,544; U.S. Pat. No. 6,451,742; and U.S. Pat. No. 6,475,745, the disclosures of which
are incorporated herein by reference. In general, the drug assays involved the addition of known concentrations of a test agent over a range of concentrations. After 72 hours, the experiment was stopped and the neurons were identified by immuno-staining. The data was expressed as % neuronal survival at I-(neuronal number is test sample/neuronal number in untreated control sample))×100%. Dose response curves were then used to estimate the effective for dose (50%) of the neuroprotective agent (defined as the ED₅₀).

Abbreviations:
DAP dapsone
HCQ hydroxychloroquine
AD Alzheimer's disease

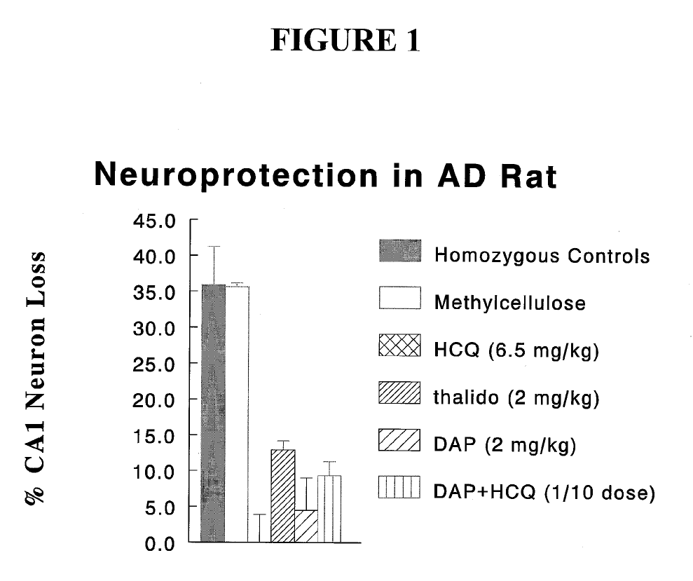

FIG. 1 is a plot of % neuron loss for several test agents (homozygous control, methylcellulose, hydroxychloroquine at a level of 6.5 mg/kg, thalidomide at a level of 2 mg/kg, dapsone at a level of 2 mg/kg, and the combination of hydroxychloroquine and dapsone at 10% of their respective individual doses, i.e., 0.65 mg/kg hydroxychloroquine+0.2 mg/kg dapsone. It is observed in FIG. 1 that in vitro hydroxychloroquine (a substituted quinoline) alone at high levels (6.5 mg/kg), dapsone (a substituted diphenyl sulfone) alone at high levels (2 mg/kg), and the combination of hydroxychloroquine (a substituted quinoline) and dapsone (a substituted diphenyl sulfone) at 10% of their respective individual doses, i.e., 0.65 mg/kg hydroxychloroquine+0.2 mg/kg dapsone produced a suppression of the in vitro killing effect.

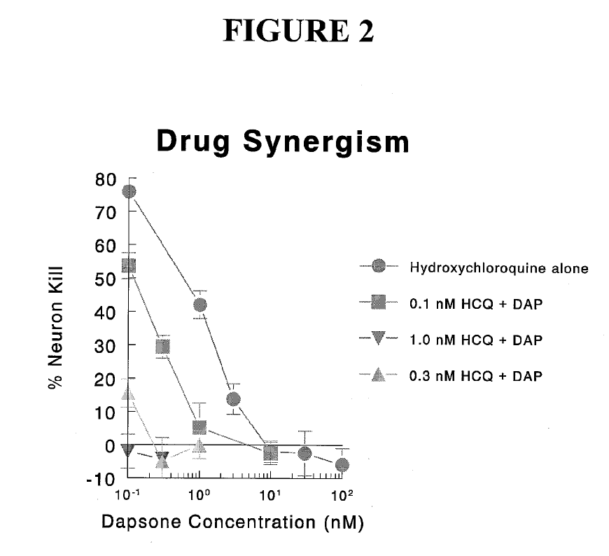

FIG. 2 is a plot of % neuron loss as a function of drug concentration for several test agents (10 nM hydroxychloroquine, the combination of 0.1 nM hydroxychloroquine+dapsone), the combination of 0.3 nM hydroxychloroquine+dapsone), the combination of 1.0 nM hydroxychloroquine+dapsone).

An isobologram is useful for determine where the effects of two agents are additive, potentiating (positively synergistic), or antagonistic (negatively synergistic). Points falling on the curved line representing a simple additive effect of the two agents, points falling in the area to the left of the curved line representing a potentiating (positively synergistic) effect of the two agents, and points falling in the area to the right of the curved line representing an antagonistic (negatively synergistic) effect of the two agents.

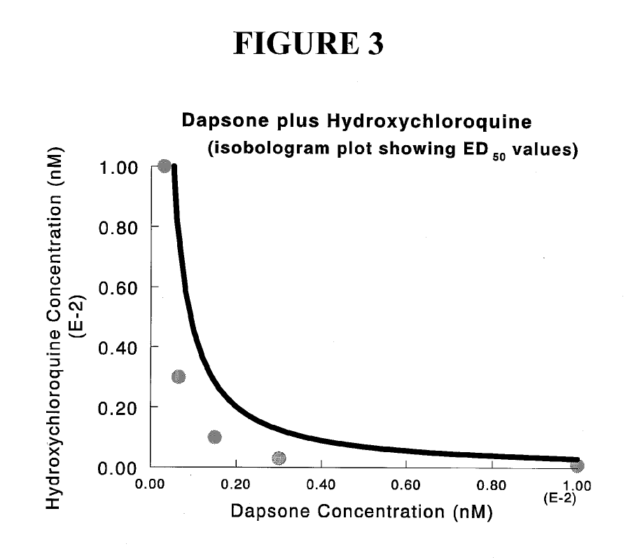

It is observed in the isobologram of FIG. 3 that, when the hydroxychloroquine (a substituted quinoline) and dapsone (a substituted diphenyl sulfone) were combined, the combination was effective at lower levels at all tested concentrations and exhibited a potentiating (positively synergistic) effect.

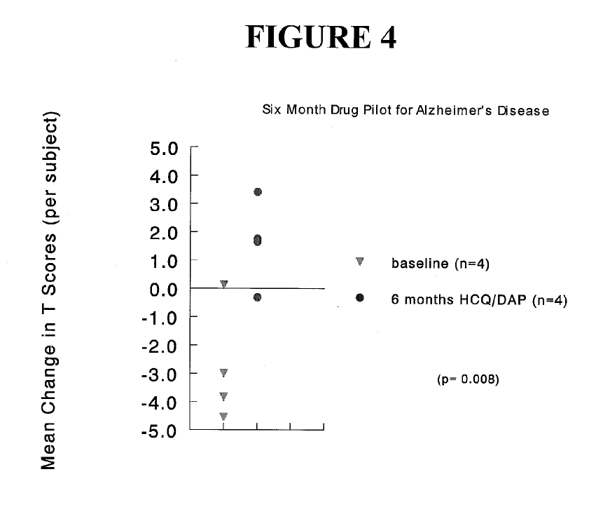

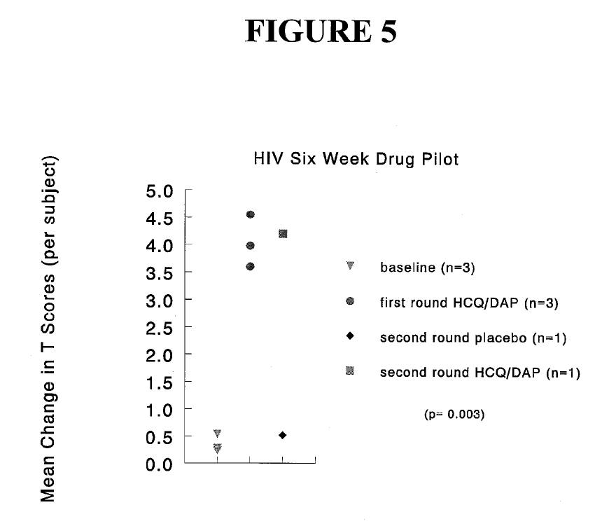

It has also been demonstrated in vivo that combinations of hydroxychloroquine (a substituted quinoline) and dapsone (a substituted diphenyl sulfone) show potentiation and achieved a reduction in neurotoxin levels in the spinal fluid of patients afflicted with Alzheimer's dementia in six-month clinical trials (as shown in FIG. 4) and of patients afflicted with HIV in a six-week clinical trial (as shown in FIG. 5). In addition, these patients were able to demonstrate an improvement in standard test with respect to cognitive function.

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating a disorder associated with neuron loss, comprising the step of:
administering to a patient in need thereof an effective amount of the composition comprising:
at least one compound of formula I: 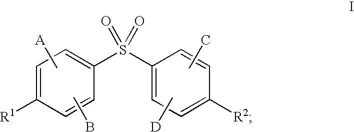
or pharmaceutically-acceptable salt or prodrug thereof;
wherein:
A, B, C, and D are, independently, H, lower alkyl, cyano, OR⁵—C(═O)OR⁵, SR⁶, halo, SO₂R⁶, NR⁶R⁷, —SO₂NR⁶R⁷;
R¹ and R² are, independently, NH₂, NHC(═O)R³, or —N═NR⁴;
R³ is H, lower alkyl, -alkyl-OR⁵, -alkyl-C(═O)OR⁵, or -alkyl-C(═O)NHR⁵;
R4 is 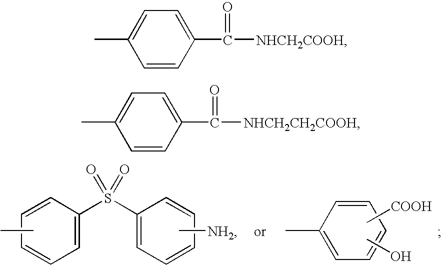
R⁵ is hydrogen, lower alkyl, substituted lower alkyl, lower alkenyl, substituted lower alkenyl, lower alkynyl, substituted lower alkynyl, aryl, haloaryl, substituted aryl, acyl, or heterocyclyl;
R⁶ is independently hydrogen, alkyl, substituted lower alkyl, lower alkenyl, substituted lower alkenyl, lower alkynyl, substituted lower alkynyl, alkyl substituted aryl, or acyl; and
R⁷ is independently hydrogen, alkyl, substituted lower alkyl, lower alkenyl, lower alkynyl, substituted lower alkenyl, substituted lower alkynyl, alkyl substituted aryl, acyl, —SO₂R⁵, or SO₂NR⁵R⁶; and
at least one compound selected from the group consisting of substituted quinoline, thalidomide, sulfasalazine, and sulfapyridine, and pharmaceutically-acceptable salts and enantiomers and prodrugs thereof;
wherein said at least one compound of formula I and said at least one second compound are administered simultaneously or sequentially in any order.

2. The method according to claim 1, wherein said disorder is Alzheimer's dementia, HIV-1 associated dementia, spongiform encephalopathy, Creutzfeld-Jakob disease, stroke, trauma, HIV infection of the central nervous system, hereditary hemorrhage with amyloidosis-Dutch type, cerebral amyloid angiopathy, or Down's syndrome.

3. The method according to claim 2, wherein said disorder is Alzheimer's dementia, HIV-1 associated dementia, or Creutzfeld-Jakob disease.

4. The method according to claim 3, wherein said disorder is Alzheimer's dementia.

5. The method according to claim 3, wherein said disorder is HIV-1 associated dementia.

6. The method according to claim 3, wherein said disorder is Creutzfeld-Jakob disease.

7. The method according to claim 1, wherein said composition is administered during the early progression of said disorder.

8. A method of treating a neurodegenerative disorder, comprising the step of:
administering to a patient in need thereof an effective amount of the composition comprising:
at least one compound of formula I: 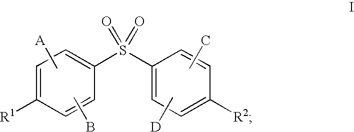
or pharmaceutically-acceptable salt or prodrug thereof;
wherein:
A, B, C, and D are, independently, H, lower alkyl, cyano, OR⁵—C(═O)OR⁵, SR⁶, halo, SO₂R⁶, NR⁶R⁷, —SO₂NR⁶R⁷;
R¹ and R² are, independently, NH₂, NHC(═O)R³, or —N═NR⁴;
R³ is H, lower alkyl, -alkyl-OR⁵, -alkyl-C(═O)OR⁵, or -alkyl-C(═O)NHR⁵;
R⁴ is 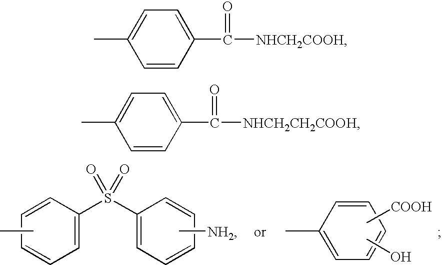
R⁵ is hydrogen, lower alkyl, substituted lower alkyl, lower alkenyl, substituted lower alkenyl, lower alkynyl, substituted lower alkynyl, aryl, haloaryl, substituted aryl, acyl, or heterocyclyl;
R⁶ is independently hydrogen, alkyl, substituted lower alkyl, lower alkenyl, substituted lower alkenyl, lower alkynyl, substituted lower alkynyl, alkyl substituted aryl, or acyl; and
R⁷ is independently hydrogen, alkyl, substituted lower alkyl, lower alkenyl, lower alkynyl, substituted lower alkenyl, substituted lower alkynyl, alkyl substituted aryl, acyl, —SO₂R⁵, or SO₂NR⁵R⁶; and
at least one compound selected from the group consisting of substituted quinoline, thalidomide, sulfasalazine, and sulfapyridine, and pharmaceutically-acceptable salts and enantiomers and prodrugs thereof;
wherein said at least one compound of formula I and said at least one second compound are administered simultaneously or sequentially in any order.

9. The method according to claim 8, wherein said neurodegenerative disorder is Alzheimer's dementia, HIV-1 associated dementia, spongiform encephalopathy, Creutzfeld-Jakob disease, stroke, trauma, HIV infection of the central nervous system, hereditary hemorrhage with amyloidosis-Dutch type, cerebral amyloid angiopathy, or Down's syndrome.

10. The method according to claim 9, wherein said neurodegenerative disorder is Alzheimer's dementia, HIV-1 associated dementia, or Creutzfeld-Jakob disease.

11. The method according to claim 10, wherein said neurodegenerative disorder is Alzheimer's dementia.

12. The method according to claim 10, wherein said neurodegenerative disorder is HIV-1 associated dementia.

13. The method according to claim 10, wherein said neurodegenerative disorder is Creutzfeld-Jakob disease.

14. The method according to claim 8, wherein said composition is administered during the early progression of said neurodegenerative disorder.

15. A method of treating a patient at risk of cognitive loss, comprising the step of:
administering to a patient in need thereof an effective amount of the composition comprising:
at least one compound of formula I: 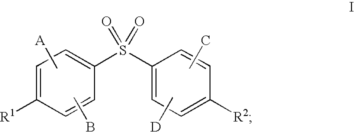
or pharmaceutically-acceptable salt or prodrug thereof,
wherein:
A, B, C, and D are, independently, H, lower alkyl, cyano, OR⁵, —C(═O)OR⁵, SR⁶, halo, SO₂R⁶, NR⁶R⁷, —SO₂NR⁶R⁷;
R¹ and R² are, independently, NH₂, NHC(═O)R³, or —N═NR⁴;
R³ is H, lower alkyl, -alkyl-OR⁵, -alkyl-C(═O)OR⁵, or -alkyl-C(═O)NHR⁵;
R⁴ is 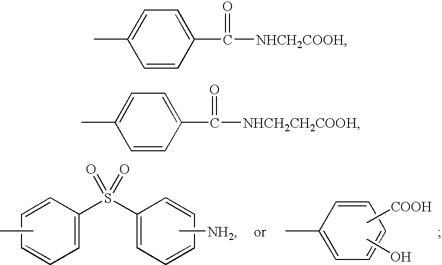
R⁵ is hydrogen, lower alkyl, substituted lower alkyl, lower alkenyl, substituted lower alkenyl, lower alkynyl, substituted lower alkynyl, aryl, haloaryl, substituted aryl, acyl, or heterocyclyl;
R⁶ is independently hydrogen, alkyl, substituted lower alkyl, lower alkenyl, substituted lower alkenyl, lower alkynyl, substituted lower alkynyl, alkyl substituted aryl, or acyl; and
R⁷ is independently hydrogen, alkyl, substituted lower alkyl, lower alkenyl, lower alkynyl, substituted lower alkenyl, substituted lower alkynyl, alkyl substituted aryl, acyl, —SO₂R⁵, or SO₂NR⁵R⁶; and
at least one compound selected from the group consisting of substituted quinoline, thalidomide, sulfasalazine, and sulfapyridine, and pharmaceutically-acceptable salts and enantiomers and prodrugs thereof;
wherein said at least one compound of formula I and said at least one second compound are administered simultaneously or sequentially in any order.

16. The method according to claim 15, wherein said patient is afflicted with mild cognitive impairment, mild cognitive motor dysfunction, HIV-associated dementia, neuro-AIDS, prion disease, acute stroke or acute trauma.

17. The method according to claim 15, wherein said composition is administered during the early progression of said cognitive loss.

18. The method according to claim 15, wherein said patient is afflicted with mild cognitive impairment.

19. The method according to claim 15, wherein said patient is afflicted with mild cognitive motor dysfunction.

20. The method according to claim 15, wherein said patient is afflicted with HIV-associated dementia or neuro-AIDS.

21. The method according to claim 15, wherein said patient is afflicted with prion disease.

22. The method according to claim 15, wherein said patient is afflicted with acute stroke or acute trauma.

23. The method according to claim 2, wherein said disorder is stroke or trauma.

24. The method according to claim 2, wherein said disorder is hereditary hemorrhage with amyloidosis-Dutch type or cerebral amyloid angiopathy.

25. The method according to claim 2, wherein said disorder is HIV-1 associated dementia or HIV infection of the central nervous system.

26. The method according to claim 2, wherein said disorder is spongiform encephalopathy or Creutzfeld-Jakob disease.

27. The method according to claim 2, wherein said disorder is Down's syndrome.

28. The method according to claim 9, wherein said neurodegenerative disorder is stroke or trauma.

29. The method according to claim 9, wherein said neurodegenerative disorder is hereditary hemorrhage with amyloidosis-Dutch type or cerebral amyloid angiopathy.

30. The method according to claim 9, wherein said neurodegenerative disorder is HIV-1 associated dementia or HIV infection of the central nervous system.

31. The method according to claim 9, wherein said neurodegenerative disorder is spongiform encephalopathy or Creutzfeld-Jakob disease.

32. The method according to claim 9, wherein said neurodegenerative disorder is Down's syndrome.

33. The method according to claim 1, 8, or 15, wherein said at least one compound of formula I or pharmaceutically-acceptable salt or prodrug thereof and said at least one second compound or pharmaceutically-acceptable salt, enantiomer, or prodrug thereof are administered simultaneously.

34. The method according to claim 1, 8, or 15, wherein said at least one compound of formula I or pharmaceutically-acceptable salt or prodrug thereof and said at least one second compound or pharmaceutically-acceptable salt, enantiomer, or prodrug thereof are administered sequentially in any order.

35. The method of claim 34, wherein said at least one compound of formula I or pharmaceutically-acceptable salt or prodrug thereof and said at least one second compound or pharmaceutically-acceptable salt, enantiomer, or prodrug thereof are administered sequentially in any order less than about one hour apart.

36. The method of claim 35, wherein said at least one compound of formula I or pharmaceutically-acceptable salt or prodrug thereof and said at least one second compound or pharmaceutically-acceptable salt, enantiomer, or prodrug thereof are administered sequentially in any order less than about 30 minutes apart.

37. The method of claim 36, wherein said at least one compound of formula I or pharmaceutically-acceptable salt or prodrug thereof and said at least one second compound or pharmaceutically-acceptable salt, enantiomer, or prodrug thereof are administered sequentially in any order less than about 5 minutes apart.
